(19) United States
(12) Patent Application Publication
(10) Pub. No.: US 2006/0035247 A1
(43) Pub. Date: Feb. 16, 2006

(54) Biochip with radio frequency identification tag attached thereto, hybridization system, and hybridization management method

(76) Inventors: Christopher Han-sung Ko, Gyeonggi-do (KR); Soo-suk Lee, Gyeonggi-do (KR); Hun-joo Lee, Seoul (KR); Gal Ju, Gyeonggi-do (KR); Joon-soo Han, Gyeonggi-do (KR)

Correspondence Address: CANTOR COLBURN, LLP, 55 GRIFFIN ROAD SOUTH, BLOOMFIELD, CT, 06002, US

(21) Appl. No.: 11/121,481

(22) Filed: May 4, 2005

(30) Foreign Application Priority Data
Aug. 12, 2004 (KR) 10-2004-0063512

(51) Int. Cl. C12Q 1/68; G06F 19/00; G01N 33/48; G01N 33/50

(52) U.S. Cl. 435/6; 702/20; 340/539.12

(57) ABSTRACT

A biochip with a radio frequency (RF) identification (ID) chip attached thereto, a hybridization system, and a hybridization management method are provided. Probes used for analyzing living body molecules are highly integrated on a substrate of the biochip, and biochip information including information obtained in the process of combining the living body molecules with the probes of the biochip is recorded in the RF ID tag of the biochip. Accordingly, it is possible to efficiently manage the biochip information using the RF ID tag and to prevent the biochip from being reused.

### BACKGROUND OF THE INVENTION

This application claims the benefit of Korean Patent Application No. 10-2004-0063512, filed on Aug. 12, 2004, in the Korean Intellectual Property Office, the disclosure of which is incorporated herein in its entirety by reference.

1. Field of the Invention

The present invention relates to a biochip, and more particularly, to a biochip, and a hybridization system and a hybridization management method which can efficiently manage information obtained during the manufacture and analysis of the biochip, can track a method in which the biochip has been used, and can prevent the biochip from being reused.

2. Description of the Related Art

Biochips, have been developed based on expertise in molecular biology and recent developments in the fields of mechanics and electronic engineering, are tiny devices into which several hundreds of or several hundreds of thousands of DNAs are implanted using mechanical automation and electronic control technologies. Biochips are classified into DNA chips or protein chips.

In the case of gene analysis using a DNA chip, samples to be analyzed are prepared. For example, pure genes are extracted from a biological sample, such as blood. Once genes are extracted, they are amplified enough to be analyzed. The amplification of the genes may be performed using a polymerase chain reaction (PCR).

Thereafter, the amplified genes (hereinafter referred to as a target sample) are hybridized with a biochip in which oligonucleotide with a known base sequence is implanted as a probe so that the target sample whose base sequence is complementary to that of the probe is combined with the probe.

Thereafter, some of the target sample that has yet to be combined with the probes is washed out from the biochip. Thereafter, the biochip is scanned using a scanner in order to determine how much of the target sample is combined with the probe. Thereafter, a resulting scanned image of each spot of the biochip is quantized and statistically analyzed.

FIG. 1 is a diagram illustrating the structure of a conventional biochip 100. Referring to FIG. 1, the conventional biochip 100 includes a substrate 120 on which probes are highly integrated and a barcode 110 which differentiates the conventional biochip 100 from other conventional biochips. Information designated by the barcode 110 is very restrictive. Therefore, detailed information on the conventional biochip 100 can be obtained by reading the barcode 110 using a barcode recognition device, such as a laser device, and then searching a server (database) based on the read barcode 110, rather than simply reading the barcode 110 using the barcode recognition device. In addition, since the barcode 110 is attached on an outer surface of the conventional biochip 100, it is highly likely to be contaminated or damaged by its surroundings.

### SUMMARY OF THE INVENTION

The present invention provides a biochip, and a hybridization system and hybridization management method which can store information regarding the biochip using a radio frequency (RF) identification (ID) tag, can also store various other information regarding sample analyzes using the biochip in the biochip, and can prevent the biochip from being reused.

According to an aspect of the present invention, there is provided a biochip including: a substrate on which probes used for analyzing living body molecules are highly integrated; a radio frequency (RF) identification (ID) tag which transmits and receives biochip information through wireless communications, the biochip information comprising actual experiment information obtained in a process of combining the living body molecules with the probes on the substrate; and a holding unit which fixes the substrate and the RF ID tag.

According to another aspect of the present invention, there is provided a hybridization system including: a biochip which comprises a substrate on which probes used for analyzing living body molecules are highly integrated, and an RF ID tag that transmits and receives predetermined data through wireless communications; and a hybridization station, which performs hybridization, washing, and drying operations on the biochip and transmits actual experiment information on each of the hybridization, washing, and drying operations to the RF ID tag of the biochip.

According to another aspect of the present invention, there is provided a hybridization management method including: (a) determining whether an RF ID tag of the biochip stores actual experiment information on a process of combining living body molecules with probes of a biochip that has been previously performed; (b) determining the biochip to have already been used and deciding not to perform a process of combining living body molecules with the probes of the biochip if the actual experiment information on the process of combining living body molecules with probes of a biochip that has been previously performed is recorded in the RF ID tag of the biochip; (c) performing the process of combining living body molecules with the probes of the biochip if the actual experiment information on the process of combining living body molecules with probes of a biochip that has been previously performed is not recorded in the RF tag of the biochip; and (d) recording actual experiment information on the process of combining living body molecules with the probes of the biochip performed in (c) in the RF ID tag of the biochip.

Accordingly, it is possible to efficiently manage information regarding a biochip, determine whether biochip-based experiments have been successfully carried out in compliance with instruction protocols, and prevent the biochip from being reused by using a RF ID tag attached to the biochip.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram illustrating the structure of a conventional biochip;

FIGS. 2 through 6 are diagrams illustrating various examples of a biochip according to an exemplary embodiment of the present invention; and

FIG. 7A is a block diagram of a hybridization system according to an exemplary embodiment of the present invention;

FIG. 7B is a block diagram of a radio frequency (RF) identification (ID) tag of a biochip of FIG. 7A;

FIG. 8 is a diagram showing a screen on which information on a biochip according to an exemplary embodiment of the present invention is displayed; and

FIG. 9 is a flowchart of a hybridization management method according to an exemplary embodiment of the present invention.

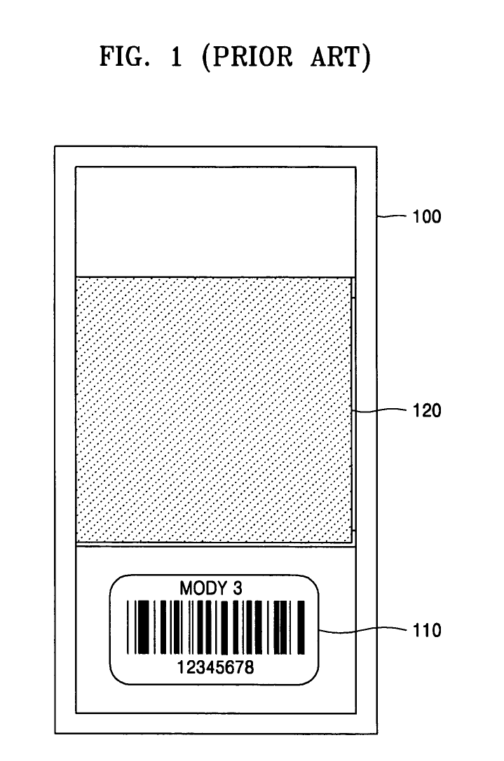

### DETAILED DESCRIPTION OF THE INVENTION

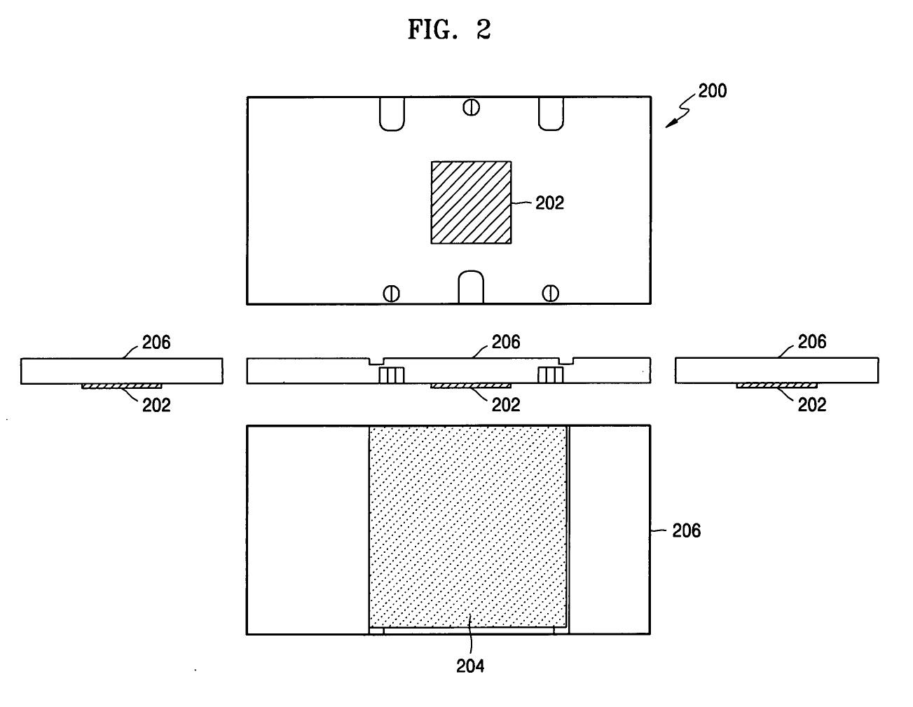

FIG. 2 is a diagram illustrating the structure of a biochip 200 according to an exemplary embodiment of the present invention. Specifically, FIG. 2 illustrates a front view, left- and right-side views, and a rear view of the biochip 200. Referring to FIG. 2, the biochip 200 includes a radio frequency (RF) identification (ID) tag 202, a substrate 204 on which probes required for analyzing living body molecules are highly integrated, and a holding unit 206 which fixes the RF ID tag 202 and the substrate 204. The RF ID tag 202 is attached on the bottom surface of the holding unit 206. The substrate 204 is attached on the top surface of the holding unit 206.

The RF ID tag 200 transmits and receives data using RF. Specifically, if the RF ID tag 202 generates a signal containing specific information, a tag reader obtains information on the RF ID tag 202 by recognizing and analyzing the signal received from the RF ID tag 202 via an antenna. The RF ID tag 200 may be classified as an active RF ID tag or a passive RF ID tag.

Information that may be recorded in the RF ID tag 202 includes: biochip manufacture information that specifies serial numbers of reagents used in the manufacture of the biochip 200, the date of manufacture of the biochip 200, the name of the manufacturer of the biochip 200, and the name(s) of the quality inspector(s) of the biochip 200; biochip chip structure information that specifies the types of probes of the biochip 200, the number of spots of the biochip 200, and the shape of the biochip 200; target sample information; information on instruction protocols (temperature, time, and speed of flow) for a process of combining a target sample with the probes of the biochip 200, which involves hybridization, washing, and drying operations; and information on conditions under which various experiments have been carried out to combine the target sample with the probes of the biochip 200.

The probes implanted into the substrate 204 of the biochip 200 are RNA, mRNA, DNA, LNA, PNA, cDNA, cRNA, protein, antibodies, and antigens.

In the present invention, hybridization may be interpreted as combination of an antigen and an antibody as well as combination of two single-stranded nucleic acids.

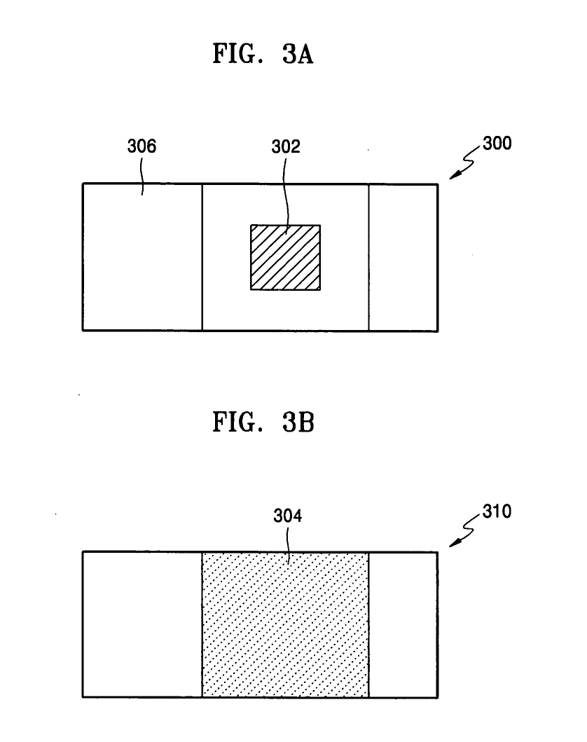

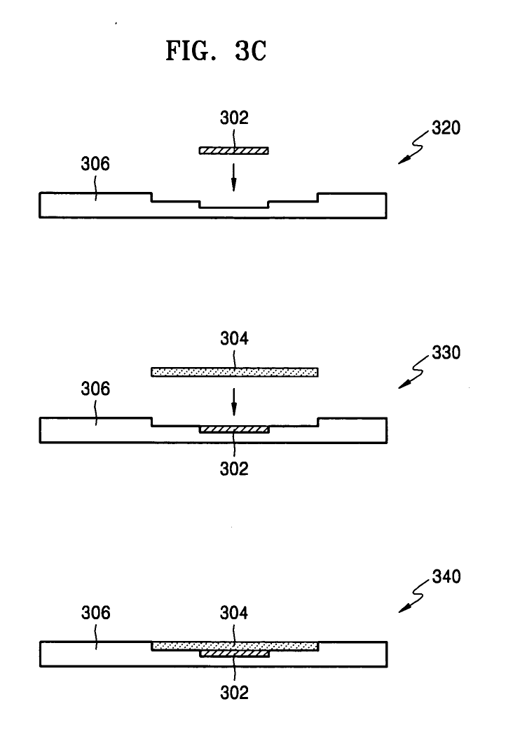

Referring to FIGS. 3A through 3C, an example of the biochip 200 of FIG. 2, i.e., a biochip 300, includes an RF ID tag 302, a substrate 304 and a holding unit 306 which has two grooves, i.e., a first groove that fits the substrate 304 and a second groove that is formed in the first groove and fits the RF ID tag 302. Accordingly, the RF ID tag 302 is installed in the second groove of the holding unit 306, and then the substrate 304 is installed in the first groove of the holding unit 306.

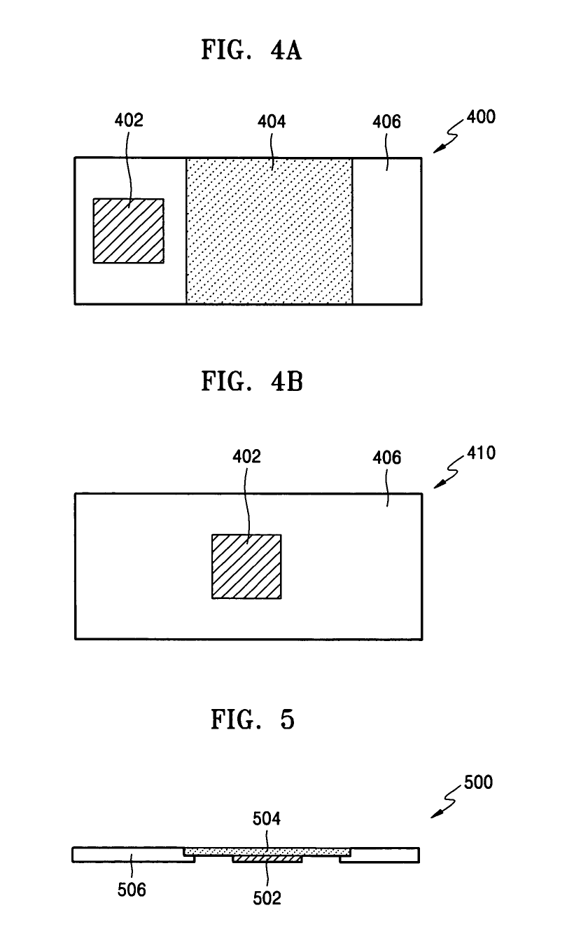

Referring to FIGS. 4A and 4B, another example of the biochip 200 of FIG. 2, i.e., a biochip 400 includes an RF ID tag 402, a substrate 404, and a holding unit 406. The RF ID tag 402 is installed at one side of the top surface or bottom surface of the holding unit 406.

Referring to FIG. 5, another example of the biochip 200 of FIG. 2, i.e., a biochip 500, includes an RF ID tag 502, a substrate 504, and holding units 506. The RF ID tag 502 is attached on the bottom surface of the substrate 504. The holding units 506 are connected to both sides of the substrate 504.

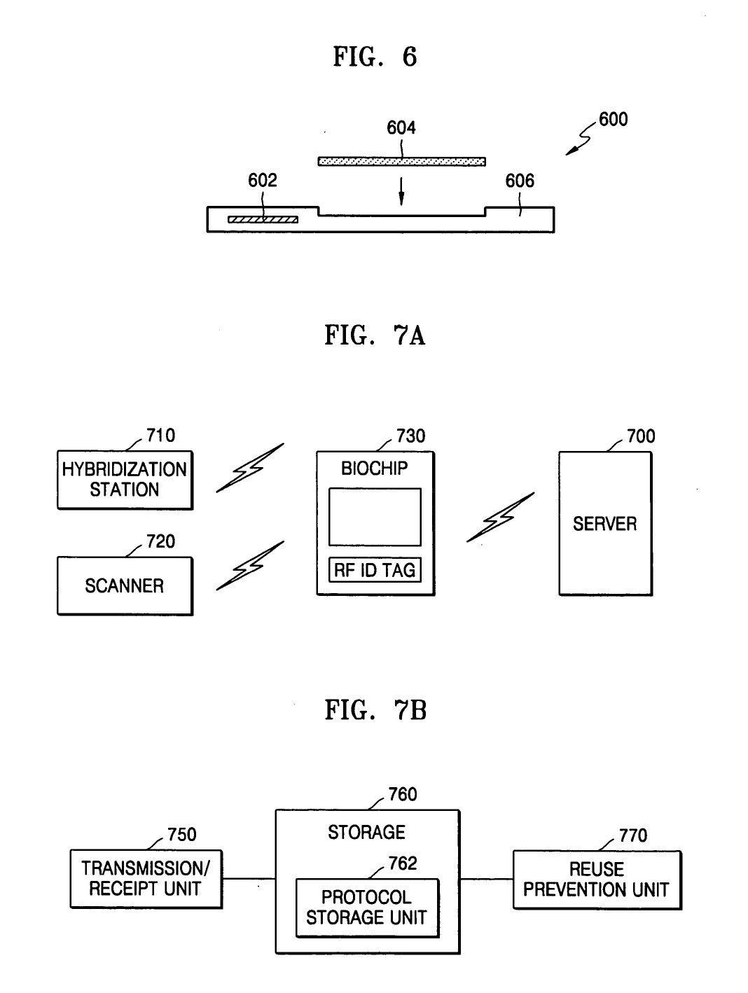

Referring to FIG. 6, another example of the biochip 200 of FIG. 2, i.e., a biochip 600, includes an RF ID tag 602, a substrate 604, and a holding unit 606. A groove is formed in the holding unit 606 so that it perfectly fits the substrate 604 when the substrate 604 is installed on the holding unit 606. The RF ID tag 602 is installed inside the holding unit 606.

The RF ID tag 202, 302, 402, 502, or 602 may be made waterproof. As described above, an RF ID tag of a biochip according to an exemplary embodiment of the present invention may be attached on the surface of a substrate or a holding unit using a sticker or an adhesive, as shown in FIGS. 2 through 5, or may be hidden inside the holding unit, as shown in FIG. 6. Therefore, the RF ID tag of the biochip according to the exemplary embodiment of the present invention can be prevented from being damaged in the process of manufacturing or inspecting the biochip according to the exemplary embodiment of the present invention.

FIG. 7A is a block diagram of a hybridization system according to an exemplary embodiment of the present invention. Referring to FIG. 7A, the hybridization system includes a hybridization station 710 and a biochip 730 having an RF ID tag. The hybridization station 710 performs hybridization, washing, and drying operations on the biochip 730. Thereafter, a scanner 720 analyzes the biochip 730 and then stores the results in the RF ID tag of the biochip 730. The RF ID tag of the biochip 730 and a server 700 may exchange data.

Management of hybridization information transmitted between the hybridization station 710 and the biochip 730 will be described in the following in further detail.

The hybridization station 710 combines living body molecules to be analyzed with a probe of the biochip 730. For example, if the biochip 730 is a DNA chip, the hybridization station 710 performs hybridization, washing, and drying operations on the biochip 730, and if the biochip 730 is a protein chip, the hybridization station 710 performs immune reaction, washing, and drying operations on the biochip 730. The hybridization (or immune reaction), washing, and drying operations are affected by such factors as time, temperature, and speed of flow. Therefore, the hybridization station 710 transmits information specifying in what order the hybridization (or immune reaction), washing, and drying operations were performed on the biochip 730, and information specifying at what temperature and speed-of-flow and how long each of the hybridization (or immune reaction), washing, and drying operations was performed to the RF ID tag of the biochip 730. If there are other conditions that may have affected the results of the hybridization (or immune reaction), washing, and drying operations, the hybridization station 710 also transmits information specifying those conditions to the RF ID tag of the biochip 730.

The hybridization station 710 may read instruction protocols (time, temperature, and speed of flow) for each of the hybridization (or immune reaction), washing, and drying operations from the RF ID tag of the biochip 730 and may determine experimental instructions appropriate for each of the hybridization (or immune reaction), washing, and drying operations. In addition, the hybridization station 710 may transmit actual experiment information regarding each of the hybridization (or immune reaction), washing, and drying operations to the RF ID tag of the biochip 730 so that the actual experiment information is recorded in the RF ID tag of the biochip 730. Then, later on, the hybridization station 710 may determine whether each of the hybridization (or immune reaction), washing, and drying operations was performed in compliance with the instruction protocols by comparing the actual experiment information with the instruction protocols.

The hybridization station 710 determines whether the actual experiment information is recorded in the RF ID tag of the biochip 730. If the actual experiment information is recorded in the RF ID tag of the biochip 730, the biochip 730 is determined to have already been used, and thus, the hybridization station 710 does not perform the hybridization (or immune reaction), washing, and drying operations on the biochip 710 in order to prevent the biochip 730 from being reused. Accordingly, it is possible to prevent various errors caused when the biochip 730 is reused.

The scanner 720 quantizes and analyzes each of the spots of the biochip 730 if the living body molecules to be analyzed are hybridized with the biochip 730 by the hybridization station 710. Specifically, the scanner 720 analyzes each of the spots of the biochip 730 in an optical manner using a laser or a CCD camera or in an electromagnetic manner using an electromagnetic field. In the case of the optical manner, the wavelength of a laser and the intensity of a photo multiplier affect the analysis of each of the spots of the biochip 730. In the case of the electromagnetic manner, the intensity of an electromagnetic field affects the analysis of each of the spots of the biochip 730.

Therefore, the scanner 720 transmits the results of the analysis of the biochip 730 and information on factors that may have affected the results of the analysis of the biochip 730, such as the wavelength of a laser and the intensity of a photo multiplier, to the RF ID tag of the biochip 730.

The RF ID tag of the biochip 730 may transmit data to and receive data from the server 700. For example, the server 700 may be a server used in a hospital in which medical information of patients who are subjected to various medical tests is recorded.

FIG. 7B is a block diagram of the RF ID tag of the biochip 730 of FIG. 7A. Referring to FIG. 7B, the RF ID tag of the biochip 730 includes a transmission/receipt unit 750, a storage unit 760, and a reuse prevention unit 770. The storage unit 760 includes a protocol storage unit 762.

The transmission/receipt unit 750 transmits data to and receives data from an external device through wireless communications. Examples of the external device include the server 700, the hybridization station 710, the scanner 720, and equipment for manufacturing the biochip 730. Once an interface required for data communication is installed in the external device, the external device can easily transmit data to or receive data from the RF ID tag of the biochip 730.

The storage unit 760 receives biochip-related information ranging from information on the manufacture of the biochip 730 to information on the hybridization and analysis of living body molecules to be analyzed from the transmission/receipt unit 750 and stores the received biochip-related information. Specifically, the storage unit 760 stores actual experiment information specifying procedures and conditions of the hybridization of the living body molecules to be analyzed with probes of the biochip 730 from the transmission/receipt unit 750. Thus, the storage unit 760 may be used for determining whether experiments for the hybridization of the living body molecules to be analyzed with the probes of the biochip 730 have been successfully carried out in compliance with instruction protocols or whether the biochip 730 has ever been used.

When the RF ID tag of the biochip 730 enters the communication range of a tag reader installed in the hybridization station 710, the hybridization station 710 reads data stored in the storage unit 760 of the RF ID tag of the biochip 730 using the tag reader and determines whether the read data is the actual experiment information. If the read data is the actual experiment information, the hybridization station 710 does not perform a hybridization operation in order to prevent the biochip 730 from being used.

The hybridization station 710 may easily determine whether the biochip 730 has ever been used based on information received from the reuse prevention unit 770 of the RF ID tag of the biochip 730.

Specifically, the reuse prevention unit 770 determines whether the actual experiment information is stored in the storage unit 760 (particularly, in the protocol storage unit 762 of the storage unit 760) and outputs a signal containing the determination results. If the hybridization station 710 receives a signal indicating that the biochip 730 has already been used from the reuse prevention unit 770, it does not perform a hybridization operation, in which case, the hybridization station 710 does not need to read the data stored in the storage unit 760. In short, the hybridization station 710 can easily determine whether the biochip 730 has ever been used based on a signal received from the reuse prevention unit 770.

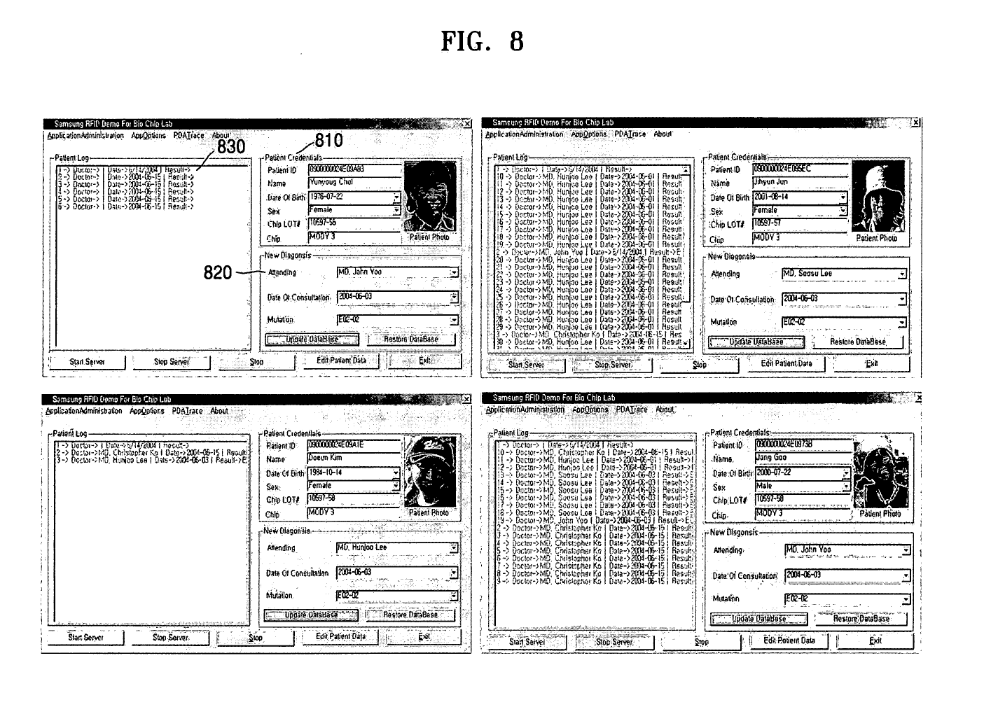

FIG. 8 is a diagram showing a screen on which information on a biochip according to an exemplary embodiment of the present invention is displayed. Referring to FIG. 8, personal records 810 of patients who are subjected to biochip-based medical tests, information 820 on the biochip-based medical tests, and results 830 of the biochip-based medical tests. The personal records 810 of the patients and the results 830 of the biochip-based medical tests are recorded in an RF ID tag of the biochip according to the exemplary embodiment of the present invention.

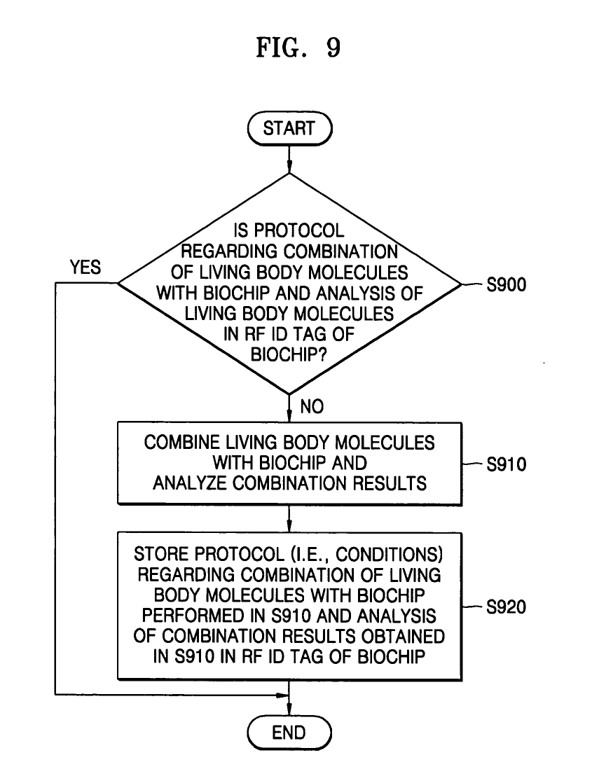

FIG. 9 is a flowchart of a hybridization management method according to an exemplary embodiment of the present invention. Referring to FIG. 9, in operation S900, information on conditions of a process of combining living body molecules to be analyzed with probes of a biochip, i.e., actual experiment information (such as time, temperature, and speed of flow), is recorded in an RF ID tag of the biochip. Here, the process of combining the living body molecules to be analyzed with the probes of the biochip involves hybridization, washing, and drying operations. If the actual experiment information is recorded in the RF ID tag of the biochip, the biochip is determined to have already been used, and thus, the process of combining the living body molecules to be analyzed with the probes of the biochip is complete. Otherwise, the process of combining the living body molecules to be analyzed with the probes of the biochip is performed and the results are analyzed under predetermined conditions in operation S910. Thereafter, in operation S920, the predetermined conditions under which the process of combining the living body molecules to be analyzed with the probes of the biochip was performed and the results were analyzed are recorded in the RF ID tag of the biochip.

According to the present invention, biochip-related information is recorded in an RF ID tag attached to a biochip, and thus, there is no need to search a database of a server for the biochip-related information.

In addition, when the RF ID tag enters a predetermined communication region, it can be recognized by and communicate with an external device. Thus, it is possible to easily transmit various information regarding the manufacture of the biochip, the combination of living body molecules to be analyzed with the biochip, and the analysis of the living body molecules combined with the biochip to the RF ID tag of the biochip through wireless communications. Moreover, it is possible to determine whether experiments have been carried out on the biochip in compliance with instruction protocols based on information recorded in the RF ID tag of the biochip. Furthermore, it is possible to prevent the biochip from being reused by determining whether the biochip has ever been used.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A biochip comprising:
a substrate on which probes used for analyzing living body molecules are highly integrated;
a radio frequency (RF) identification (ID) tag which transmits and receives biochip information through wireless communications, the biochip information comprising actual experiment information obtained in a process of combining the living body molecules with the probes on the substrate; and
a holding unit which fixes the substrate and the RF ID tag.

2. The biochip of claim 1, wherein the RF ID tag transmits and receives biochip manufacture information that specifies a process of fixing the probes to the substrate, instruction protocols for the process of combining the living body molecules with the probes of the biochip, actual experiment information that specifies experimenters, procedures and conditions of the process of combining the living body molecules with the probes on the substrate, information on target samples, information on preparation of the target samples, and results of analyzing the biochip.

3. The biochip of claim 1, wherein if the actual experiment information is recorded in the RF ID tag, the RF ID tag generates a signal indicating that the process of combining living body molecules with the probes of the biochip will not be allowed to be performed.

4. The biochip of claim 1, wherein the holding unit is rectangular and has a first groove in which the substrate is installed and a second groove in which the RF ID tag is installed, wherein the second groove is formed in the first groove.

5. The biochip of claim 1, wherein the RF ID tag is attached on one surface of the substrate, and the holding unit is attached to both sides of the substrate.

6. The biochip of claim 1, wherein the holding unit is rectangular and has a groove in which the substrate is installed, and the RF ID tag is inserted inside the holding unit.

7. A hybridization system comprising:
a biochip which comprises a substrate on which probes used for analyzing living body molecules are highly integrated, and an RF ID tag that transmits and receives predetermined data through wireless communications; and
a hybridization station, which performs hybridization, washing, and drying operations on the biochip and transmits actual experiment information on each of the hybridization, washing, and drying operations to the RF ID tag of the biochip.

8. The hybridization system of claim 7, wherein the hybridization station determines whether the actual experiment information is recorded in the RF ID tag of the biochip and performs the hybridization, washing, and drying operations on the biochip if the actual experiment information is not recorded in the RF ID tag of the biochip.

9. A hybridization management method comprising:
(a) determining whether an RF ID tag of the biochip stores actual experiment information on a process of combining living body molecules with probes of a biochip that has been previously performed;
(b) determining the biochip to have already been used and deciding not to perform a process of combining living body molecules with the probes of the biochip if the actual experiment information on the process of combining living body molecules with probes of a biochip that has been previously performed is recorded in the RF ID tag of the biochip;
(c) performing the process of combining living body molecules with the probes of the biochip if the actual experiment information on the process of combining living body molecules with probes of a biochip that has been previously performed is not recorded in the RF tag of the biochip; and
(d) recording actual experiment information on the process of combining living body molecules with the probes of the biochip performed in (c) in the RF ID tag of the biochip.

10. The hybridization management method of claim 9, wherein the actual experiment information specifies the duration, temperature, and speed of flow of each of the hybridization, washing, and drying operations performed in the process of combining living body molecules with the probes of the biochip that has been previously performed or has been performed in (c).
